(19) United States
(12) Patent Application Publication
(10) Pub. No.: US 2007/0178570 A1
(43) Pub. Date: Aug. 2, 2007

(54) SUSTAINED MICROBIAL PRODUCTION OF HYDROGEN GAS FROM DILUTED FRUIT JUICE

(76) Inventor: Harry R. Diz, Erie, PA (US)

Correspondence Address: Gwen R. Acker Wood, Ph.D., J.D.;Eckert Seamans Cherin & Mellott, LLC, 44th Floor, 600 Grant Street, Pittsburgh, PA, 15219, US

(21) Appl. No.: 11/668,707

(22) Filed: Jan. 30, 2007

Related U.S. Application Data
(60) Provisional application No. 60/764,292, filed on Feb. 1, 2006.

(51) Int. Cl. C12P 3/00; C12M 3/00

(52) U.S. Cl. 435/168; 435/289.1

(57) ABSTRACT

The present invention provides an apparatus and method for the production of hydrogen based on the capture of metabolic by-products of hydrogen-producing microbacteria, in which a bioreactor is maintained in an environment conducive to the growth of hydrogen-producing microbacteria and the production of hydrogen and at the same time is restrictive to the growth of undesirable microorganisms such as methanogens and the production of methane. The present invention utilizes concentrated growth of hydrogen-producing microbacteria such as Klebsiella oxytoca. The invention provides a simple and cost-effective way to produce hydrogen by selectively harnessing hydrogen-producing microbacteria utilizing glucose-based solutions while substantially eliminating methane-producing microbacteria.

### CROSS REFERENCE TO RELATED APPLICATIONS

The present application claims priority to U.S. Provisional Patent Application No. 60/764,292, filed Feb. 1, 2006, which is incorporated by reference herein.

### FIELD OF THE INVENTION

The present invention relates generally to a method and apparatus for concentrated production of hydrogen-generating microbacteria cultures. More particularly, this invention relates to a method and apparatus for the biological production of hydrogen while substantially avoiding the production of methane, the invention utilizing concentrated growth of hydrogen-producing microbacteria such as Klebsiella oxytoca. The invention provides a simple and cost-effective way to produce hydrogen by selectively harnessing hydrogen-producing microbacteria utilizing glucose-based solutions while substantially eliminating methane-producing microbacteria.

### BACKGROUND OF THE INVENTION

The production of hydrogen is an increasingly common and important procedure in the world today. Production of hydrogen in the United States alone currently amounts to about 3 billion cubic feet per year, with output likely to increase. Uses for the produced hydrogen are varied, ranging from uses in welding, in production of hydrochloric acid, and for reduction of metallic ores. An increasingly important use of hydrogen is in fuel cells or for combustion. This is directly related to the production of alternative fuels for machinery such as motor vehicles. Successful use of hydrogen as an alternative fuel can provide substantial benefits to the world at large. This is possible not only because hydrogen is produced without dependence on the location of specific oils or other ground resources, but because burning hydrogen is atmospherically clean. Essentially, no carbon dioxide or greenhouse gasses are produced when burning hydrogen. Thus, production of hydrogen as a fuel source can have a great impact on decreasing the use of fuels that produce greenhouse gases.

Production of hydrogen from various methods generally is known. For example, electrolysis, which basically involves the use of electricity to decompose water into hydrogen and oxygen, is a commonly used process. Significant energy, however, is required to produce the needed electricity to perform the process. Similarly, steam reforming is another expensive method requiring fossil fuels as an energy source. As can be readily understood, the environmental benefits of producing hydrogen are at least partially offset when using a process that requires pollution-causing fuels as an energy source for the production of hydrogen.

Thus, production of hydrogen from biological systems, wherein the energy for the process is substantially provided by naturally occurring bacteria, is an optimal solution. Fermentation of organic matter by hydrogen-producing microbacteria, such as Bacillus or Clostridium, is one such method. However, substantial and useful production of hydrogen gas from microbacteria is problematic. The primary obstacle to sustained production of useful quantities of hydrogen by microbacteria has been the eventual stoppage of hydrogen production generally coinciding with the appearance of methane. This occurs when methanogenic bacteria invade the reactor environment, converting hydrogen to methane. This process occurs naturally in anaerobic environments such as marshes, swamp and pond sediments. As the appearance of methanogens in a biological system previously has been largely inevitable, continuous production of hydrogen from hydrogen-producing microbacteria has been unsuccessful in the past.

New methods of hydrogen generation that optimize yields of hydrogen while minimizing expenditures, therefore, are needed. One possible method is to produce hydrogen in a biological system by converting organic matter into hydrogen gas. The production of biogas that is substantially hydrogen theoretically can be achieved in a bioreactor, in which hydrogen-producing microbacteria and an organic source solution are held in an environment favorable to hydrogen production.

Microbiologists have known for many years that certain microorganisms produce hydrogen as a metabolic by-product. Two reviews of this body of knowledge are Kosaric and Lyng (1988) and Nandi and Sengupta (1998). Among the various organisms mentioned, the heterotrophic facultative anaerobes are of interest in this study, particularly those in the group known as enteric bacteria. Within this group are the mixed-acid fermentors, whose most well known member is Escherichia coli. While fermenting glucose, these bacteria split the glucose molecule, forming two moles of pyruvate, an acetyl group is stripped from each pyruvate fragment leaving formic acid, which then is cleaved into equal amounts of carbon dioxide and hydrogen. Thus, during this process, one mole of glucose produces two moles of hydrogen gas. Also produced during this process are acetic and lactic acids, and minor amounts of succinic acid and ethanol. Other enteric bacteria (the 2, 3 butanediol fermentors) use a different enzyme pathway which causes additional CO₂ generation resulting in a 6:1 ratio of carbon dioxide to hydrogen production (Madigan et al., 1997).

There are many sources of waste organic matter which could serve as a substrate for this microbial process, namely as a provider of pyruvate. One such attractive material would be organic-rich industrial wastewaters, particularly sugar-rich waters, such as fruit and vegetable processing wastes. Other sources include agricultural residues and other organic wastes such as sewage and animal manures.

It is of further importance to increase the number of hydrogen-producing microorganisms in a system to the point that a fixed colony is existent. Increasing the number of hydrogen-producing microbacteria and thereby increasing the overall percentage of hydrogen-producing microbacteria is beneficial, particularly in large scale reactors. Therefore, it is important to create a bioreactor environment that is conducive to hydrogen-producing microbacterial growth and maintenance in addition to hydrogen production.

Thus, there exists a need to produce substantial and useful levels of hydrogen in an inexpensive, environmentally friendly manner utilizing hydrogen-producing microbacteria.

### SUMMARY OF THE INVENTION

The present invention fulfills this need by providing an apparatus and method for the production of hydrogen based on the capture of metabolic by-products of hydrogen-producing microbacteria, in which a bioreactor is maintained in an environment conducive to the growth of hydrogen-producing microbacteria and the production of hydrogen and at the same time is restrictive to the growth of undesirable microorganisms such as methanogens and the production of methane.

It is an object of the present invention to provide an apparatus and method for producing hydrogen from hydrogen-producing microbacteria that metabolize organic feed material which includes a bioreactor for receiving organic feed material and adapted to produce hydrogen from the hydrogen-producing microbacteria metabolizing the organic feed material, and a pH controller in operable relation to the bioreactor, wherein the pH controller can adjust the pH of the organic feed material in the system, wherein the pH controller is set to control the pH of the organic feed material to a range of about 3.5-6.0.

It is a further object of the present invention to provide an apparatus and method for producing hydrogen from hydrogen-producing microbacteria that metabolize organic feed material which includes a bioreactor for receiving organic feed material and adapted to produce hydrogen from the hydrogen-producing microbacteria metabolizing the organic feed material, a heater for heating the organic feed material prior to introduction into the bioreactor, and a pH controller in operable relation to the bioreactor, wherein the pH controller can adjust the pH of the organic feed material in the system.

It is a further object of the present invention to provide an apparatus and method wherein the heater heats the organic feed material to a temperature of about 60° C. to 100° C.

These and other objects of the present invention will become more readily apparent from the following detailed description and appended claims.

### BRIEF DESCRIPTION OF THE DRAWINGS

FIG. 1 is a plan view of the hydrogen production apparatus.

### DETAILED DESCRIPTION OF PREFERRED EMBODIMENTS

As used herein, the term “microbacteria” include bacteria and substantially microscopic cellular organisms.

As used herein, the term “hydrogen-producing microbacteria” includes microorganisms that metabolize an organic substrate in one or a series of reactions that ultimately forms hydrogen as one of the end products.

As used herein, the term “methanogens” refers to microbacteria that metabolize hydrogen in one or a series of reactions that produces methane as one of the end products.

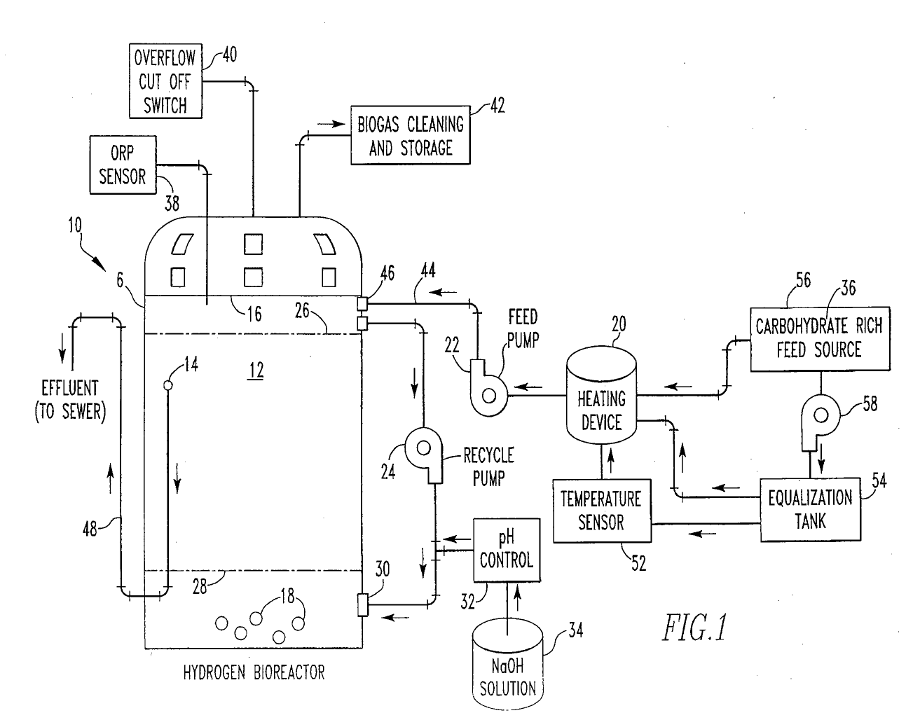

A hydrogen-producing bioreactor 10 for sustained production of hydrogen in accordance with the present invention is shown in FIG. 1. The bioreactor 10 is anaerobic and therefore substantially airtight. The bioreactor 10 may contain several openings. However, these openings are covered with substantially airtight coverings or connections, thereby keeping the environment in the bioreactor 10 substantially anaerobic. The bioreactor 10 can be any receptacle known in the art for carrying an aqueous solution 12. Generally, the receptacle will be a limiting factor in the amount of hydrogen that can be produced. The larger the receptacle, the more aqueous solution containing hydrogen-producing bacteria, and, by extension, hydrogen, can be produced. Therefore, the size and shape of the bioreactor can vary widely within the spirit of the invention depending on the output of hydrogen desired and location limitations. In the present embodiment depicted in FIG. 1, the bioreactor 10 holds a volume of about 2 liters of aqueous solution. The embodiment of the invention, however, can be readily scaled to a much larger volume.

The bioreactor 10 can be formed of any material suitable for holding an aqueous solution and that can create an airtight, anaerobic environment. In the present invention, the bioreactor 10 is constructed of acrylic materials. Other suitable materials include, for example and without limitation, metals and other plastics. Furthermore, the bioreactor 10 preferably is formed to substantially prevent the entry of air therein.

To maintain the aqueous solution volume level at a general constant level, the bioreactor 10 preferably provides means to remove excess solution. In the present embodiment, the bioreactor 10 includes an effluent tube 48 that extends from inside the bioreactor 10 to outside the bioreactor 10 and into a suitable location for effluent, such as a sewer. Inside the bioreactor 10, the effluent tube 48 extends upwards to a surface level 16 that generally coincides to a level that is a preferred top surface area of the solution if the bioreactor 10 is filled. The effluent tube 48 has an open end 14 at or near the surface level 16. When the bioreactor 10 is filled, the open end of the effluent tube 48 allows a gravity overflow to maintain a constant fluid volume. The bioreactor 10 may further contain a retaining plate 26 and perforated acrylic plate 28.

The bioreactor 10 contains one or a multiplicity of substrates 18 for providing surface area for attachment and growth of bacterial biofilms. The sizes and shapes of the substrates 18 can vary widely, including, but not limited to, flat surfaces, pipes, rods, beads, slats, tubes, slides, screens, honeycombs, spheres, objects with latticework, or other objects with holes bored through the surface.

Substrates 18 preferably are substantially free of small interior spaces that potentially fill with gas. In the present embodiment, the bioreactor 10 contains substrates 18 comprising about 150 pieces of floatable one inch plastic media to provide surface area for attachment of the bacterial biofilm. This includes, but is not limited to, Flexiring™ Random Packing (Koch-Glitsch.) The packing may be retained below the liquid surface by the perforated acrylic plate 28.

In an embodiment of the present invention, organic feed solution 36 first is contained in a reservoir 56. The reservoir 56 is a container known in the art that can contain an organic feed solution. The size, shape and material of the reservoir 56 can vary widely within the spirit of the invention. In one embodiment, the reservoir 56 is one or a multiplicity of storage tanks that are adaptable to receive, hold and store the organic feed solution 36 when not in use, wherein the one or a multiplicity of storage tanks may be mobile. In another embodiment, the reservoir 56 is a wastewater well that is adaptable to receive and contain wastewater and/or effluent from an industrial process. In a further embodiment, the reservoir 56 is adaptable to receive and contain wastewater which is effluent from a juice manufacturing industrial process, such that the effluent held in the reservoir 56 is a sugar rich juice sludge.

The organic feed solution 36 in the reservoir 56 is conveyed throughout the apparatus, such that the apparatus is a closed system of continuous movement. Conveyance of organic feed solution 36 can be achieved by any conveying means known in the art, for example, one or a multiplicity of pumps. The apparatus of the present invention uses a closed system, such that a few well placed conveying means can convey the organic feed solution 36 throughout the apparatus, from the reservoir 56 to an optional equalization tank 54 to the heating device 20 to the bioreactor 10 to outside of the bioreactor 10. In an embodiment, the organic feed solution 36 contained in the reservoir 56 is conveyed into the optional equalization tank 54 with a pump 58. The pump 58 is in operable relation to the reservoir 56 such that it aids removal movement of organic feed solution 36 to the equalization tank 54 at a desired, adjustable flow rate, wherein the pump 58 can be any pump known in the art suitable for pumping liquids. In a preferred embodiment, the pump 58 is a submersible sump pump.

The organic feed solution 36 leaving the reservoir 56 can be conveyed either into the equalization tank 54 or the heating device 20. The equalization tank 54 is an optional intermediary container for holding the organic feed solution 36 between the reservoir 56 and the heating device 20. The equalization tank 54 provides an intermediary container that can help control the flow rates of organic feed solution 36 into the heating device 20 by providing a slower flow rate out of the equalization tank 54 than the flow rate of organic feed solution into the equalization tank 54. An equalization tank is most useful when the reservoir 56 receives effluent from an industrial facility such that it is difficult to control flow into the reservoir 56. The equalization tank can be formed of any material suitable for holding and treating the organic feed solution 36. In the present invention, the equalization tank 54 is constructed of acrylic materials. Other materials include, but are not limited to, metals or non-acrylic plastics. Additionally, the size and shape of the equalization tank 54 can vary widely within the spirit of the invention depending on output desired and location limitations.

The organic feed solution is heated prior to conveyance into the bioreactor to deactivate or kill methane producing microorganisms, i.e., methanogens. The heating can occur anywhere upstream. In one embodiment, the heating is achieved in the heating device 20, wherein the organic feed solution 36 is heated within the heating device. Alternatively, organic feed solution can be heated at additional or alternate locations in the hydrogen production system.

In a preferred embodiment, the feed source 36 is a grape juice solution prepared using Welch's Concord Grape Juice™ diluted in tap water at approximately 32 ml of juice per liter. The grape juice solution is inoculated with one or a multiplicity of hydrogen-producing bacteria in an inoculation step. The added hydrogen-producing microbacteria may include the same types of microbacteria that occur naturally in organic-rich industrial wastewaters. In an embodiment, the hydrogen-producing microbacteria added in an inoculation step are microbacteria that thrive in pH levels of about 3.5 to 6.0 and can survive at elevated temperatures. These hydrogen-producing microbacteria include, but are not limited to, Clostridium sporogenes, Bacillus licheniformis and Kleibsiella oxytoca. The grape juice solution is aerated for 24 hours to substantially remove any chlorine. Due to the acidity of the juice, the pH of the feed solution typically is about 4.0. The constitutional make-up of the grape juice solution is shown in Table 1.

TABLE 1
Composition of concord grape juice. Source:
Welch's Company, personal comm., 2005.

Concentration (unit indicated)

| Constituent | Mean | Range |
|---|---|---|
| Carbohydrates¹ | | 15–18% |
| glucose | 6.2% | 5–8% |
| fructose | 5.5% | 5–8% |
| sucrose | 1.8% | 0.2–2.3% |
| maltose | 1.9% | 0–2.2% |
| sorbitol | 0.1% | 0–0.2% |
| Organic Acids¹ | | 0.5–1.7% |
| Tartaric acid | 0.84% | 0.4–1.35% |
| Malic acid | 0.86% | 0.17–1.54% |
| Citric acid | 0.044% | 0.03–0.12% |
| Minerals¹ | | |

| | | |
|---|---|---|
| Calcium | 17–34 | mg/L |
| Iron | 0.4–0.8 | mg/L |
| Magnesium | 6.3–11.2 | mg/L |
| Phosphorous | 21–28 | mg/L |
| Potassium | 175–260 | mg/L |
| Sodium | 1–5 | mg/L |
| Copper | 0.10–0.15 | mg/L |
| Manganese | 0.04–0.12 | mg/L |
| Vitamins¹ | | |
| Vitamin C | 4 | mg/L |
| Thiamine | 0.06 | mg/L |
| Riboflavin | 0.04 | mg/L |
| Niacin | 0.2 | mg/L |
| Vitamin A | 80 | I.U. |

| | |
|---|---|
| pH | 3.0–3.5 |
| Total solids | 18.5% |

¹additional trace constituents in these categories may be present.

In a preferred embodiment, the organic feed solution 36 is heated in the heating device 20 and conveyed into the bioreactor 10 with a feed pump 22 at a desired flow rate through a conveying means 44 and inlet 46. The heating unit 20 can elevate the temperature of the solution in the bioreactor 10 between a range of about 60° C. to about 100° C. A heating temperature of about 60° C.-70° C. for at least 30 minutes temporarily deactivates methanogens in a pasteurization-like process. Heating temperatures of between about 90° C.-100° C. for at least thirty minutes can destroy methanogens. In contrast, many hydrogen-producing microbacteria are resistant to temperatures up to about 110° C. for over three hours. The heating device 20 enables heating of the organic feed solution 36 to a temperature of about 60° C.-110° C. to substantially deactive or kill the methanogens while leaving any hydrogen-producing microbacteria substantially functional. In a preferred embodiment, the temperature of the feed source 36 is elevated in the heating unit 20 to about 65° C.

The heating device 20 can be any suitable receptacle known in the art for holding, receiving and conveying the feed source 36. Preferably, the heating device 20 is formed substantially from metals, acrylics, other plastics or combinations thereof, yet the material can vary widely within the spirit of the invention to include other suitable materials. Similarly, the size and shape of the heating device 20 can vary widely within the spirit of the invention depending on the output required and location limitations. In preferred embodiments, retention time in the heating device 20 is at least one hour. Retention time marks the average time that any particular part of the feed source 36 is retained in the heating device 20.

To maintain the temperatures at desired levels, at least one temperature sensor 52 monitors a temperature indicative of the feed source 36 temperature, preferably the temperature level of the heating device 20. In preferred embodiments, an electronic controller is provided having at least one microprocessor adapted to process signals from one or a plurality of devices providing feed source 36 parameter information, wherein the electronic controller is operably related to at least one actuatable terminal and is arranged to control the operation of and to controllably heat the heating device 20 and/or any contents therein. The electronic controller is located or coupled to the heating device 20, or can alternatively be at a third or remote location. In alternate embodiments, the controller for controlling the temperature of the heating device 20 is not operably related to the temperature sensor 52, and temperatures can be adjusted manually in response to temperature readings taken from the temperature sensor 52.

The feed pump 22 pumps the feed source 36 through the conveying means 44, through the inlet 46 and into the bioreactor 10. The inlet 46 generally can be located at any location along the bioreactor 10. However, in preferred embodiments, the inlet 46 is located near the upper portion of the bioreactor 10 below the surface level 16 and above the retaining plate 26. The conveying means can be any apparatus known in the art, most typically a tube. Once inside the bioreactor 10, the feed source 36 becomes solution.

The bioreactor 10 preferably is equipped with a recycle pump 24 to rapidly recirculate solution drawn at or near the top of the bioreactor 10 to the lower levels of the bioreactor 10. In one embodiment, the recycle pump 24 takes solution from above a retaining plate 26 and reintroduces the solution through a deflector fitting 30 into the bottom of the reactor, resulting in a dispersed vertical flow of liquid. The up-flow circulation aids microbacteria within the solution to find glucose sources on which to grow biofilms.

The pH level of the solution is controlled within the bioreactor 10. Precise control of a pH level provides an environment that enables at least some hydrogen-producing bacteria to function while similarly providing an environment unfavorable to methanogens. This enables the concept of allowing microbacterial reactions to create hydrogen without subsequently being overrun by methanogens that convert the hydrogen to methane. This produces a biological system in the bioreactor 10 without substantial methane production.

Control of pH of the solution in the bioreactor can be achieved by any means known in the art. In the present embodiments, a pH control monitoring device 32 monitors the pH and can add a pH control solution in an automated manner if the pH of the solution in the bioreactor moves out of a desired range. In a preferred embodiment, the pH monitor controls the bioreactor solution's pH through automated addition of a 0.2 M NaOH solution 34. One such apparatus for achieving this is an Etatron DLX pH monitoring device. Preferred ranges of pH for the bioreactor solution is between about 3.5 and 6.5, with a more preferred range between about 4.0 and 5.5.

The bioreactor 10 preferably further includes an oxidation-reduction potential (ORP) probe 38 which allows the monitoring of redox potential of the solution, an overflow cut-off switch 40 to turn off the feed pump 22 if the solution exceeds a certain level in the bioreactor and a device for capturing the hydrogen. In the present embodiment, the hydrogen is conveyed from the bioreactor 10 through a passage 42 and captured in bags 50 made of materials specifically designed to contain hydrogen (SKC, Inc.). After capture, the hydrogen gas may be further treated, cleaned and/or stored.

The organic feed solution preferably is carbohydrate-rich to better enable the hydrogen production reaction. There are many organic sources which can be utilized as a feed source. These include, for example, agricultural residues, industrial wastes and other organic material such as sewage or manure. Examples of organic source material include, without limitation, organic-rich industrial wastewaters such as fruit and vegetable processing wastes or juices. In a preferred embodiment, the solution introduced into the receptacle is a high sugar organic waste, for example and without limitation, sludge or other waste or by-products that are resultant from the production of a fruit juice, such as grape juice. In additional embodiments, wastewaters rich not only in sugars but also in protein and fats can be used, such as milk product wastes. The most complex potential source of energy for this process would include sewage-related wastes, such as municipal sewage and animal manures. Typical hydrogen-producing microbacteria are adept at metabolizing the high sugar organic waste into bacterial waste products

Hydrogen-producing microbacteria metabolize the sugars in the organic feed solution under the reactions:

Glucose→2 Pyruvate (1)

2 Pyruvate+2 Coenzyme A→2 Acetyl-CoA+2 HCOOH (2)

2 HCOOH→2H₂+2 CO₂ (3)

During this process, one mole of glucose produces two moles of hydrogen gas and carbon dioxide. In alternate embodiments, other organic feed solutions include agricultural residues and other organic wastes such as sewage and manures. Typical hydrogen-producing microbacteria are adept at metabolizing the high sugar organic waste into bacterial waste products. The wastewater may be further treated by aeration, diluting the solution with water or other dilutants, adding compounds that control the pH of the solution or other treatment steps. For example, the electrolyte contents (Na, K, Cl, Mg, Ca, etc.) of the organic feed solution can be adjusted. Further, the solution may be supplemented with phosphorus (NaH₂PO₄) or yeast extract.

The organic feed solution of the present invention provides a plentiful feeding ground for hydrogen-producing microbacteria which is naturally infested with these microorganisms. While hydrogen-producing microbacteria typically occur naturally in an organic feed solution, the organic feed solution of the present invention preferably is further inoculated with hydrogen-producing microbacteria in an inoculation step. In further preferred embodiments, the inoculation is an initial, one-time addition to the bioreactor at the beginning of the hydrogen production process. The initial inoculation provides enough hydrogen-producing microbacteria to produce sustained colonies of hydrogen-producing microbacteria within the bioreactor. The sustained colonies allow the sustained production of hydrogen. Further inoculations of hydrogen-producing microbacteria may be added as desired. The added hydrogen-producing microbacteria may include the same types of microorganisms that occur naturally in the organic feed solution. In preferred embodiments, the hydrogen-producing microbacteria, whether occurring naturally or added in an inoculation step, are preferably microorganisms that thrive in pH levels of about 3.5 to 6.0 and can survive in temperatures of 60° F.-110° F. or, more preferably, 60° F.-75°. These hydrogen-producing microbacteria include, but are not limited to, Clostridium sporogenes, Bacillus licheniformis and Kleibsiella oxytoca. Hydrogen-producing microbacteria can be obtained from a microorganism culture laboratory or like source. Other hydrogen-producing microbacteria or hydrogen-producing microbacteria known in the art, however, can be used within the spirit of the invention. The inoculation step can occur in the bioreactor or elsewhere in the apparatus.

Although hydrogen-producing microbacteria typically occur naturally in the organic feed solution, the solution in the bioreactor may be inoculated further with one or a multiplicity of hydrogen-producing bacteria in an inoculation step. The inoculation may be an initial, one-time addition to the bioreactor at the beginning of the hydrogen production process. Further inoculations, however, may be added as desired. The added hydrogen-producing microbacteria may include the same types of microbacteria that occur naturally in the feed source. In preferred embodiments, the hydrogen-producing microbacteria, whether occurring naturally or added in an inoculation step, are microbacteria that thrive in pH levels of about 3.5 to 6.0 and can survive at elevated temperatures. These hydrogen-producing microbacteria include, but are not limited to, Clostridium sporogenes, Bacillus licheniformis and Kleibsiella oxytoca. Hydrogen-producing microbacteria can be obtained from a bacterial culture laboratory or like source.

In an embodiment, the preferred hydrogen-producing bacteria are Kleibsiella oxytoca, a facultative enteric bacterium capable of hydrogen generation. Kleibsiella oxytoca may be obtained from a source such as yeast extract. In this embodiment, the continuous input of seed organisms from the yeast extract in the feed source results in a culture of Kleibsiella oxytoca in the bioreactor solution. Alternatively, the bioreactor may be directly inoculated with Kleibsiella oxytoca. In this embodiment, the inoculum for each bioreactor is about 100 ml (of a 48 hour culture in nutrient broth) added to 1.9 L of diluted grape juice, with each bioreactor operating in batch mode for one day. The bioreactors need not be stripped of oxygen before or after inoculation. The ORP is monitored with the ORP sensor 38. Once ORP drops below about −200 mV, gas production commences. Subsequently, when operating in a continuous flow mode, the ORP typically is in the range of about −300 to −450 mV.

The volume of collected gas can be determined by water displacement before and after scrubbing with concentrated NaOH. Samples of scrubbed and dried gas may be analyzed for hydrogen and methane by gas chromatography with a thermal conductivity detector (TCD) and/or with a flame ionization detector (FID). Both hydrogen and methane produce a response in the TCD, but the response to methane is improved in the FID (hydrogen is not detected by an FID, which uses hydrogen as a fuel for the flame). Pure hydrogen gas is can be obtained from a tank of ultra high purity hydrogen and methane can be obtained from a laboratory gas cock.

The present invention also provides a method for producing hydrogen from organic solutions comprised of providing an organic feed source solution, heating the organic feed source solution with a heating device, conveying the organic feed source solution into a bioreactor, inoculating the organic feed source solution with one or a multiplicity of hydrogen-producing microbacteria to produce a microbacteria solution, monitoring the microbacteria solution with a pH monitoring device which is capable of introducing a compound, such as, for example and without limitation, NaOH, into the microbacteria solution if the pH of the microbacteria solution moves out of a desired range, creating an upstream movement of the microbacteria solution in the bioreactor and capturing the hydrogen produced in a suitable device typically known in the art.

The present invention is more particularly described in the following non-limiting example, which is intended to be illustrative only, as numerous modifications and variations therein will be apparent to those skilled in the art.

### EXAMPLE 1

A multiplicity of bioreactors initially were operated at pH 4.0 and a flow rate of 2.5 mL min⁻¹, resulting in a hydraulic retention time (HRT) of about 13 h (0.55 d). This is equivalent to a dilution rate of 1.8 d⁻¹. After one week, all six bioreactors were at pH 4.0, the ORP ranged from −300 to −450 mV, total gas production averaged 1.6 L d⁻¹ and hydrogen production averaged 0.8 L d⁻¹. The mean chemical oxygen demand (COD) of the organic feed material during this period was 4,000 mg L⁻¹ and the mean effluent COD was 2,800 mg L⁻¹, for a reduction of 30%. After one week, the pHs of certain bioreactors were increased by one half unit per day until the six bioreactors were established at different pH levels ranging from 4.0 to 6.5. Over the next three weeks at the new pH settings, samples were collected and analyzed each weekday. It was found that the optimum for gas production in this embodiment was pH 5.0 at 1.48 L hydrogen d⁻¹) (Table 2). This was equivalent to about 0.75 volumetric units of hydrogen per unit of bioreactor volume per day.

TABLE 2
Production of hydrogen in 2-L anaerobic bioreactors as a function
of pH.

| | Total | | H2 | H2 per |
|---|---|---|---|---|
| | gas | H2 | L/g | Sugar |
| pH | L/day | L/day | COD | moles/mole |
| 4.0^{a} | 1.61 | 0.82 | 0.23 | 1.81 |
| 4.5^{b} | 2.58 | 1.34 | 0.23 | 1.81 |
| 5.0^{c} | 2.74 | 1.48 | 0.26 | 2.05 |
| 5.5^{d} | 1.66 | 0.92 | 0.24 | 1.89 |
| 6.0^{d} | 2.23 | 1.43 | 0.19 | 1.50 |
| 6.5^{e} | 0.52 | 0.31 | 0.04 | 0.32 |

^{a}mean of 20 data points
^{b}mean of 14 data points
^{c}mean of 11 data points
^{d}mean of 7 data points
^{e}mean of 6 data points

Also shown in Table 2 is the hydrogen production rate per g of COD, which also peaked at pH 5.0 at a value of 0.26 L g⁻¹ COD consumed. To determine the molar production rate, it was assumed that each liter of hydrogen gas contained 0.041 moles, based on the ideal gas law and a temperature of 25° C. Because most of the nutrient value in the grape juice was simple sugars, predominantly glucose and fructose (Table 1 above), it was assumed that the decrease in COD was due to the metabolism of glucose. Based on the theoretical oxygen demand of glucose (1 mole glucose to 6 moles oxygen), one gram of COD is equivalent to 0.9375 g of glucose. Therefore, using those conversions, the molar hydrogen production rate as a function of pH ranged from 0.32 to 2.05 moles of hydrogen per mole of glucose consumed. As described above, the pathway appropriate to these microorganisms results in two moles of hydrogen per mole of glucose, which was achieved at pH 5.0. The complete data set is provided in Tables 3a and 3b.

TABLE 3a
Bioreactor Operating Data

| | | | GAS | | | | | | |
|---|---|---|---|---|---|---|---|---|---|
| | | | Tot | after | Liquid | | | | |
| | | collection | volume | scrubbing | Effluent | NaOH | Net Feed | Readings | |
| Date | Reactor | hours | (mL) | (mL) | (mL) | (mL) | (mL) | ORP | pH |
| 17-Nov | C | 5.5 | 360 | 200 | 840 | 120 | 720 | −344 | 4.9 |
| 18-Nov | C | 5 | 370 | 200 | 1120 | 70 | 1050 | −328 | 4.9 |
| 29-Nov | C | 4.25 | 415 | 200 | 920 | 50 | 870 | −403 | 4.9 |
| 17-Nov | E | 5.5 | 490 | 270 | 1210 | 115 | 1095 | −352 | 5.0 |
| 1-Dec | D | 3.5 | 540 | 250 | 710 | 85 | 625 | −395 | 5.0 |
| 17-Nov | F | 5.5 | 475 | 225 | 1120 | 130 | 990 | −367 | 5.0 |
| 5-Dec | D | 4.5 | 580 | 310 | 710 | 77 | 633 | −423 | 5.0 |
| 6-Dec | D | 3 | 450 | 240 | 490 | 43 | 447 | −420 | 5.0 |
| 17-Nov | D | 3.5 | 680 | 415 | 580 | 83 | 497 | −326 | 5.0 |
| 2-Dec | D | 3.75 | 640 | 340 | 830 | 66 | 764 | −412 | 5.0 |
| 22-Nov | C | 3.75 | 460 | 295 | 800 | 50 | 750 | −349 | 5.0 |
| averages | | 4.34 | 496 | 268 | 848 | 81 | 767 | −374.5 | 5.0 |
| 5-Dec | C | 4.5 | 470 | 250 | 900 | 103 | 797 | −429 | 5.4 |
| 18-Nov | F | 5 | 90 | 45 | 600 | 55 | 545 | −451 | 5.5 |
| 21-Nov | D | 4 | 130 | 70 | 830 | 80 | 750 | −454 | 5.5 |
| 22-Nov | D | 3.75 | 360 | 250 | 765 | 68 | 696 | −461 | 5.5 |
| 29-Nov | D | 4.25 | 100 | 50 | 940 | 100 | 840 | −456 | 5.5 |
| 2-Dec | C | 3.75 | 550 | 290 | 810 | 93 | 717 | −430 | 5.5 |
| 6-Dec | C | 3 | 250 | 130 | 570 | 45 | 525 | −428 | 5.5 |
| averages | | 4.04 | 279 | 155 | 774 | 78 | 696 | −444.1 | 5.5 |
| 21-Nov | E | 4 | 350 | 250 | 930 | 130 | 800 | −400 | 6.0 |
| 22-Nov | E | 3.75 | 380 | 280 | 820 | 127 | 693 | −411 | 6.0 |
| 29-Nov | E | 4.25 | 360 | 230 | 870 | 71 | 799 | −467 | 6.0 |
| 1-Dec | E | 3.5 | 420 | 250 | 770 | 127 | 643 | −471 | 6.0 |
| 2-Dec | E | 3.75 | 280 | 170 | 540 | 85 | 455 | −443 | 6.0 |
| 5-Dec | E | 4.5 | 410 | 240 | 930 | 156 | 774 | −487 | 6.0 |
| 6-Dec | E | 3 | 280 | 170 | 660 | 105 | 555 | −490 | 6.0 |
| averages | | 3.82 | 354 | 227 | 789 | 114 | 674 | −453 | 6.0 |
| 29-Nov | F | 4.25 | 90 | 45 | 870 | 150 | 720 | −501 | 6.5 |
| 2-Dec | F | 3.75 | 20 | 0 | 810 | 136 | 674 | −497 | 6.5 |
| 22-Nov | F | 3.75 | 120 | 105 | 790 | 128 | 662 | −477 | 6.5 |
| 5-Dec | F | 4.5 | 10 | 0 | 670 | 121 | 549 | −532 | 6.5 |
| 6-Dec | F | 3 | 60 | 50 | 480 | 90 | 390 | −515 | 6.5 |
| 21-Nov | F | 4 | 200 | 100 | 910 | 150 | 760 | −472 | 6.5 |
| averages | | 3.88 | 83 | 50 | 755 | 129 | 626 | −499 | 6.5 |

| | COD | | | | | Performance | | |
|---|---|---|---|---|---|---|---|---|
| | Feed | Effluent | Removal | Loading | Consumed | Total gas | H2 | H2 |
| Date | (mg/L) | (mg/L) | (mg/L) | (g) | (g) | L/day | L/day | L/g COD |
| 17-Nov | 4,907 | 2,880 | 2,027 | 3.533 | 1.459 | 1.57 | 0.87 | 0.14 |
| 18-Nov | 3,680 | 2,480 | 1,200 | 3.864 | 1.260 | 1.78 | 0.96 | 0.16 |
| 29-Nov | 5,013 | 3,093 | 1,920 | 4.362 | 1.670 | 2.34 | 1.13 | 0.12 |
| 17-Nov | 4,907 | 4,747 | 160 | 5.373 | 0.175 | 2.14 | 1.18 | 1.54 |
| 1-Dec | 6,173 | 3,573 | 1,600 | 3.233 | 1.000 | 3.70 | 1.71 | 0.25 |
| 17-Nov | 4,907 | 3,760 | 1,147 | 4.858 | 1.135 | 2.07 | 0.98 | 0.20 |
| 5-Dec | 4,267 | 3,573 | 694 | 2.701 | 0.439 | 3.09 | 1.65 | 0.71 |
| 6-Dec | 4,853 | 3,253 | 1,600 | 2.169 | 0.715 | 3.60 | 1.92 | 0.34 |
| 17-Nov | 4,907 | 4,213 | 694 | 2.439 | 0.345 | 4.66 | 2.85 | 1.20 |
| 2-Dec | 4,587 | 3,787 | 800 | 3.504 | 0.611 | 4.10 | 2.18 | 0.56 |
| 22-Nov | 4,107 | 1,280 | 2,827 | 3.080 | 2.120 | 2.94 | 1.89 | 0.14 |
| averages | 4,664 | 3,331 | 1,333 | 3.579 | 1.023 | 2.74 | 1.48 | 0.26 |
| 5-Dec | 4,267 | 3,413 | 854 | 3.401 | 0.680 | 2.51 | 1.33 | 0.37 |
| 18-Nov | 3,680 | 3,440 | 240 | 2.006 | 0.131 | 0.43 | 0.22 | 0.34 |
| 21-Nov | 3,493 | 3,360 | 133 | 2.620 | 0.100 | 0.78 | 0.42 | 0.70 |
| 22-Nov | 4,107 | 2,880 | 1,227 | 2.858 | 0.854 | 2.30 | 1.60 | 0.29 |
| 29-Nov | 5,013 | 3,307 | 1,707 | 4.211 | 1.434 | 0.56 | 0.28 | 0.03 |
| 2-Dec | 4,587 | 3,573 | 1,014 | 3.289 | 0.727 | 3.52 | 1.86 | 0.40 |
| 6-Dec | 4,853 | 3,627 | 1,226 | 2.548 | 0.644 | 2.00 | 1.04 | 0.20 |
| averages | 4,286 | 3,371 | 914 | 2.982 | 0.636 | 1.66 | 0.92 | 0.24 |
| 21-Nov | 3,493 | 2,987 | 506 | 2.794 | 0.406 | 2.10 | 1.50 | 0.62 |
| 22-Nov | 4,107 | 2,453 | 1,653 | 2.846 | 1.146 | 2.43 | 1.79 | 0.24 |
| 29-Nov | 5,013 | 1,973 | 3,040 | 4.006 | 2.429 | 2.03 | 1.30 | 0.09 |
| 1-Dec | 5,173 | 2,933 | 2,240 | 3.326 | 1.440 | 2.88 | 1.71 | 0.17 |
| 2-Dec | 4,587 | 3,360 | 1,227 | 2.087 | 0.558 | 1.79 | 1.09 | 0.30 |
| 5-Dec | 4,267 | 3,253 | 1,014 | 3.303 | 0.785 | 2.19 | 1.28 | 0.31 |
| 6-Dec | 4,853 | 2,293 | 2,560 | 2.693 | 1.421 | 2.24 | 1.36 | 0.12 |
| averages | 4,499 | 2,750 | 1,749 | 3.033 | 1.179 | 2.23 | 1.43 | 0.19 |
| 29-Nov | 5,013 | 1,707 | 3,307 | 3.610 | 2.381 | 0.51 | 0.25 | 0.02 |
| 2-Dec | 4,587 | 3,573 | 1,014 | 3.092 | 0.683 | 0.13 | 0.00 | 0.00 |
| 22-Nov | 4,107 | 2,240 | 1,867 | 2.719 | 1.236 | 0.77 | 0.67 | 0.08 |
| 5-Dec | 4,267 | 2,827 | 1,440 | 2.343 | 0.791 | 0.05 | 0.00 | 0.00 |
| 6-Dec | 4,853 | 2,240 | 2,613 | 1.893 | 1.019 | 0.48 | 0.40 | 0.05 |
| 21-Nov | 3,493 | 2,613 | 880 | 2.655 | 0.669 | 1.20 | 0.60 | 0.15 |
| averages | 4,387 | 2,533 | 1,853 | 2.745 | 1.160 | 0.52 | 0.31 | 0.04 |

TABLE 3b
Bioreactor Operating Data Continued.

| | | | Gas | | | | | | |
|---|---|---|---|---|---|---|---|---|---|
| | | | Total | after | Liquid | | | | |
| | | collection | volume | scrubbing | Effluent | NaOH | Net Feed | Readings | |
| Date | Reactor | hours | (mL) | (mL) | (mL) | (mL) | (mL) | ORP | pH |
| 14-Nov | A | 5 | 540 | 220 | 780 | 0 | 780 | −408 | 4.0 |
| 14-Nov | B | 5 | 380 | 220 | 840 | 0 | 840 | −413 | 4.1 |
| 14-Nov | C | 5 | 350 | 170 | 870 | 0 | 870 | −318 | 4.1 |
| 14-Nov | D | 5 | 320 | 130 | 920 | 0 | 920 | −372 | 4.1 |
| 14-Nov | E | 5 | 240 | 100 | 920 | 0 | 920 | −324 | 4.3 |
| 14-Nov | F | 5 | 50 | 25 | 810 | 0 | 810 | −329 | 4.0 |
| 15-Nov | A | 5.5 | 450 | 230 | 1120 | 25 | 1095 | −400 | 4.0 |
| 15-Nov | B | 5.5 | 450 | 235 | 1180 | 35 | 1145 | −384 | 4.0 |
| 15-Nov | C | 5.5 | 250 | 130 | 640 | 0 | 640 | −278 | 4.0 |
| 15-Nov | E | 5.5 | 455 | 225 | 1160 | 0 | 1160 | −435 | 4.0 |
| 15-Nov | F | 5.5 | 430 | 235 | 1160 | 0 | 1160 | −312 | 4.0 |
| 16-Nov | A | 5 | 380 | 190 | 1020 | 27 | 993 | −414 | 4.0 |
| 5-Dec | A | 4.5 | 200 | 110 | 500 | 35 | 465 | −439 | 4.0 |
| 18-Nov | A | 5 | 360 | 190 | 200 | 0 | 200 | −423 | 4.0 |
| 21-Nov | A | 4 | 320 | 170 | 800 | 40 | 760 | −429 | 4.0 |
| 22-Nov | A | 3.75 | 285 | 190 | 725 | 21 | 704 | −432 | 4.0 |
| 29-Nov | A | 4.25 | 310 | 155 | 750 | 24 | 726 | −439 | 4.0 |
| 2-Dec | A | 3.75 | 250 | 120 | 660 | 26 | 634 | −438 | 4.0 |
| 6-Dec | A | 3 | 150 | 75 | 540 | 0 | 540 | −441 | 4.0 |
| 17-Nov | A | 5.5 | 300 | 160 | 1010 | 30 | 980 | −414 | 4.0 |
| averages | | 4.81 | 324 | 164 | 830 | 13 | 817 | −392 | 4.0 |
| 16-Nov | B | 5 | 400 | 200 | 1125 | 45 | 1080 | −397 | 4.5 |
| 16-Nov | D | 5 | 400 | 165 | 960 | 60 | 900 | −360 | 4.5 |
| 16-Nov | E | 5 | 490 | 240 | 1100 | 72 | 1028 | −324 | 4.5 |
| 1-Dec | B | 3.5 | 500 | 260 | 570 | 45 | 525 | −415 | 4.5 |
| 6-Dec | B | 3 | 470 | 240 | 650 | 40 | 610 | −411 | 4.5 |
| 21-Nov | B | 4 | 560 | 300 | 930 | 50 | 880 | −397 | 4.5 |
| 2-Dec | B | 3.75 | 640 | 320 | 830 | 50 | 780 | −407 | 4.5 |
| 17-Nov | B | 5.5 | 450 | 220 | 1165 | 50 | 1115 | −406 | 4.5 |
| 18-Nov | B | 5 | 390 | 220 | 860 | 42 | 818 | −406 | 4.5 |
| 22-Nov | B | 3.75 | 585 | 395 | 835 | 50 | 785 | −397 | 4.5 |
| 29-Nov | B | 4.25 | 620 | 320 | 920 | 42 | 878 | −410 | 4.5 |
| 5-Dec | B | 4.5 | 390 | 190 | 750 | 37 | 713 | −417 | 4.5 |
| 16-Nov | F | 5 | 400 | 200 | 1082 | 96 | 989 | −324 | 4.5 |
| 16-Nov | C | 5 | 400 | 200 | 950 | 74 | 876 | −325 | 4.6 |
| averages | | 4.45 | 478 | 248 | 909 | 54 | 856 | −385 | 4.5 |

| | COD | | | | | Performance | | |
|---|---|---|---|---|---|---|---|---|
| | Feed | Effluent | Removal | Loading | Consumed | Total gas | H2 | H2 |
| Date | (mg/L) | (mg/L) | (mg/L) | (g) | (g) | L/day | L/day | L/g COD |
| 14-Nov | 4,480 | 2,293 | 2,187 | 3.494 | 1.706 | 2.59 | 1.06 | 0.13 |
| 14-Nov | 4,480 | 2,453 | 2,027 | 3.763 | 1.702 | 1.82 | 1.06 | 0.13 |
| 14-Nov | 4,480 | 2,293 | 2,187 | 3.896 | 1.902 | 1.68 | 0.82 | 0.09 |
| 14-Nov | 4,480 | 1,920 | 2,560 | 4.122 | 2.355 | 1.54 | 0.62 | 0.06 |
| 14-Nov | 4,480 | 2,773 | 1,707 | 4.122 | 1.570 | 1.15 | 0.48 | 0.06 |
| 14-Nov | 3,307 | 2,080 | 1,227 | 2.679 | 0.994 | 0.24 | 0.12 | 0.03 |
| 15-Nov | 3,307 | 3,787 | (480) | 3.621 | −0.525 | 1.96 | 1.00 | −0.44 |
| 15-Nov | 3,307 | 3,253 | 54 | 3.787 | 0.061 | 1.96 | 1.03 | 3.82 |
| 15-Nov | 3,307 | 3,520 | (213) | 2.116 | −0.136 | 1.09 | 0.57 | −0.95 |
| 15-Nov | 3,307 | 3,467 | (160) | 3.836 | −0.185 | 1.99 | 0.96 | −1.21 |
| 15-Nov | 3,307 | 3,413 | (106) | 3.836 | −0.123 | 1.88 | 1.03 | −1.91 |
| 16-Nov | 4,693 | 3,627 | 1,066 | 4.660 | 1.059 | 1.82 | 0.91 | 0.18 |
| 5-Dec | 4,267 | 4,160 | 107 | 1.984 | 0.050 | 1.07 | 0.59 | 2.21 |
| 18-Nov | 3,680 | 5,227 | (1,547) | 0.736 | −0.309 | 1.73 | 0.91 | −0.61 |
| 21-Nov | 3,493 | 3,680 | (187) | 2.655 | −0.142 | 1.92 | 1.02 | −1.20 |
| 22-Nov | 4,107 | 2,293 | 1,813 | 2.891 | 1.277 | 1.82 | 1.22 | 0.15 |
| 29-Nov | 5,013 | 3,520 | 1,493 | 3.640 | 1.084 | 1.75 | 0.88 | 0.14 |
| 2-Dec | 4,587 | 3,893 | 694 | 2.906 | 0.440 | 1.60 | 0.77 | 0.27 |
| 6-Dec | 4,853 | 3,093 | 1,760 | 2.621 | 0.950 | 1.20 | 0.60 | 0.08 |
| 17-Nov | 4,907 | 3,520 | 1,387 | 4.809 | 1.359 | 1.31 | 0.70 | 0.12 |
| averages | 4,092 | 3,213 | 879 | 3.344 | 0.718 | 1.61 | 0.82 | 0.23 |
| 16-Nov | 4,693 | 3,520 | 1,173 | 5.068 | 1.267 | 1.92 | 0.96 | 0.16 |
| 16-Nov | 4,693 | 3,573 | 1,120 | 4.224 | 1.008 | 1.92 | 0.79 | 0.16 |
| 16-Nov | 4,693 | 3,413 | 1,280 | 4.824 | 1.315 | 2.35 | 1.15 | 0.18 |
| 1-Dec | 5,173 | 3,680 | 1,493 | 2.716 | 0.784 | 3.43 | 1.78 | 0.33 |
| 6-Dec | 4,853 | 3,360 | 1,493 | 2.960 | 0.911 | 3.76 | 1.92 | 0.26 |
| 21-Nov | 3,493 | 3,147 | 346 | 3.074 | 0.305 | 3.36 | 1.80 | 0.98 |
| 2-Dec | 4,587 | 3,413 | 1,174 | 3.578 | 0.915 | 4.10 | 2.05 | 0.35 |
| 17-Nov | 4,907 | 2,933 | 1,974 | 5.471 | 2.201 | 1.96 | 0.96 | 0.10 |
| 18-Nov | 3,680 | 2,960 | 720 | 3.010 | 0.589 | 1.87 | 1.06 | 0.37 |
| 22-Nov | 4,107 | 2,720 | 1,387 | 3.224 | 1.089 | 3.74 | 2.53 | 0.36 |
| 29-Nov | 5,013 | 3,307 | 1,707 | 4.402 | 1.496 | 3.50 | 1.81 | 0.21 |
| 5-Dec | 4,267 | 3,840 | 427 | 3.042 | 0.304 | 2.08 | 1.01 | 0.62 |
| 16-Nov | 4,693 | 3,093 | 1,600 | 4.641 | 1.582 | 1.92 | 0.96 | 0.13 |
| 16-Nov | 4,693 | 2,933 | 1,760 | 4.111 | 1.541 | 1.92 | 0.96 | 0.13 |
| averages | 4,539 | 3,278 | 1,261 | 3.883 | 1.079 | 2.58 | 1.34 | 0.23 |

Samples of biogas were analyzed several times per week from the beginning of the study, initially using a Perkin Elmer Autosystem GC with TCD, and then later with a Perkin Elmer Clarus 500 GC with TCD in series with an FID. Methane was never detected with the TCD, but trace amounts were detected with the FID (as much as about 0.05%).

Over a ten-day period, the organic feed material was mixed with sludge obtained from a methane-producing anaerobic digester at a nearby wastewater treatment plant at a rate of 30 mL of sludge per 20 L of diluted grape juice. There was no observed increase in the concentration of methane during this period. Therefore, it was concluded that the preheating of the feed to 65° C. as described previously was effective in deactivating the microorganisms contained in the sludge. Hydrogen gas production rate was not affected.

Using this example, hydrogen gas was generated using a microbial culture over a sustained period of time. The optimal pH for this culture consuming simple sugars from a simulated fruit juice bottling wastewater was found to be 5.0. Under these conditions, using plastic packing material to retain microbial biomass, a hydraulic residence time of about 0.5 days resulted in the generation of about 0.75 volumetric units of hydrogen gas per unit volume of bioreactor per day.

Whereas particular embodiments of this invention have been described above for purposes of illustration, it will be evident to those skilled in the art that numerous variations of the details of the present invention may be made without departing from the invention as defined in the appended claims.

We claim:

1. An apparatus for producing hydrogen from an organic solution, comprising:
a bioreactor;
one or a multiplicity of substrates within the bioreactor for the formation of one or a multiplicity of microbacterial biofilms on the one or multiplicity of substrates;
a conveying means connected to the bioreactor, the conveying means able to convey an organic feed solution into the bioreactor through a passage;
a heating device for heating the organic feed solution;
a pH control monitoring device in operable relation to the bioreactor;
a recycle pump in operable relation to the bioreactor; and
a hydrogen capturing device on an upper portion of the bioreactor for capturing hydrogen produced by the apparatus.

2. The apparatus of claim 1, wherein the bioreactor contains an aqueous solution having one or a multiplicity of hydrogen-producing microbacteria therein.

3. The apparatus of claim 1, wherein the one or multiplicity of hydrogen-producing microbacteria is selected from the group consisting of Clostridium sporogenes, Bacillus licheniformis and Kleibsiella oxytoca.

4. The apparatus of claim 1, wherein the one or multiplicity of substrates are comprised of a plurality of pieces of floatable plastic media to provide surface area for attachment of the one or multiplicity of microbacterial biofilms.

5. The apparatus of claim 1, wherein the organic feed solution is subjected to pretreatment selected from the group consisting of aeration, dilution with water or other diluents, addition of compounds to control pH and supplementation with phosphorus or yeast extract.

6. The apparatus of claim 1, wherein the organic feed solution is a grape juice solution comprising grape juice diluted with tap water and aerated for a period of time to substantially remove any chlorine therein.

7. The apparatus of claim 1, wherein the heating device heats the organic feed solution to a temperature of about 60° C. to 100° C.

8. The apparatus of claim 1, wherein the apparatus further includes a temperature sensor for monitoring the temperature of the organic feed solution in the heating device.

9. The apparatus of claim 1, wherein the pH monitoring device controls the pH of the organic solution in the bioreactor through automated addition of a sodium hydroxide solution.

10. The apparatus of claim 1, wherein the pH monitoring device maintains the pH of the organic solution in the bioreactor in a range of about 3.5-6.0.

11. The apparatus of claim 1, wherein the bioreactor further includes an ORP sensor.

12. The apparatus of claim 1, wherein the bioreactor further includes a recycle pump to recirculate the solution in the bioreactor in an up-flow circulation pattern.

13. The apparatus of claim 1, wherein the bioreactor further includes a retaining plate in the upper portion of the bioreactor and a deflector fitting in the bottom of the reactor.

14. The apparatus of claim 1, wherein the apparatus further includes an effluent passage providing removal access from the bioreactor.

15. A method for producing hydrogen from organic solutions, comprising:
providing an organic feed source solution;
heating the organic feed source solution with a heating device;
conveying the organic feed source solution into a bioreactor;
inoculating the organic feed source solution with one or a multiplicity of hydrogen-producing microbacteria to produce a microbacteria solution;
monitoring the microbacteria solution with a pH monitoring device, wherein the pH monitoring device introduces a compound into the microbacterial solution if the pH of the microbacteria solution moves out of a desired range;
creating an upstream movement of the microbacteria solution in the bioreactor; and
capturing the produced hydrogen in a capturing device.

16. The method of claim 15, wherein the organic feed source solution is a grape juice solution comprising grape juice diluted with tap water and aerated for a period of time to substantially remove any chlorine therein.

17. The method of claim 15, wherein the heating device heats the organic feed source solution to a temperature of about 60° C. to 100° C.

18. The method of claim 15, wherein the temperature of the organic feed source solution in the heating device is monitored using a temperature sensor device.

19. The method of claim 15, wherein the pH monitoring device controls the pH of the organic feed solution in the bioreactor through automated addition of a sodium hydroxide solution.

20. The method of claim 15, wherein the pH monitoring device maintains the pH of the organic feed solution in the bioreactor in a range of about 3.5-6.0.

21. The method of claim 15, wherein the one or multiplicity of hydrogen-producing microbacteria is selected from the group consisting of Clostridium sporogenes, Bacillus licheniformis and Kleibsiella oxytoca.
